(19) United States
(12) Patent Application Publication
(10) Pub. No.: US 2026/0094697 A1
(43) Pub. Date: Apr. 2, 2026

(54) SYSTEMS AND METHODS FOR FACILITATING CLINICAL TRAINING, UPSKILLING, RESKILLING, AND CLINICAL SITE PLACEMENT

(71) Applicant: Elaina McAdams, Moorpark, CA (US)

(72) Inventor: Elaina McAdams, Moorpark, CA (US)

(21) Appl. No.: 18/904,191

(22) Filed: Oct. 2, 2024

Related U.S. Application Data
(60) Provisional application No. 63/536,869, filed on Sep. 6, 2023.

(51) Int. Cl. G16H 40/20

(52) U.S. Cl. CPC G16H 40/20

(57) ABSTRACT

A system and method for a clinical education transition coordinator/preceptor system configured for cloud-based clinical assessment, training, and competency management of users. The system may include a shared marketplace or consortium, including resources relevant to specific geographic clinical education markets. The system may include a clinical education transition coordinator/preceptor application configured for training individuals in order to reskill and upskill licensed clinicians as well as for providing clinical site support for clinical students during clinical rotations.

### CROSS-REFERENCE TO RELATED APPLICATIONS

The present application claims priority to U.S. Provisional Application No. 63/536,869 filed Sep. 6, 2023, titled “COLLEGE AND UNIVERSITY HEALTHCARE EDUCATION CONSORTIUM TRANSITION TO CLINICAL PRACTICE UPSKILLING AND RESKILLING MODEL,” which is hereby incorporated by reference in its entirety.

### TECHNICAL FIELD

The embodiments generally relate to systems and methods for facilitating the transition to practice model of reskilling and upskilling of licensed clinicians and clinical students or students majoring in health science fields.

### BACKGROUND

Currently, there is no true bidirectional sharing of clinical resources in the healthcare space or the academic space. There are state hospital associations, professional clinical organizations, vendor management systems, etc., that have been created to share best practices, but they are only able to address issues at a surface level due to conflict of interest.

### SUMMARY

This summary is provided to introduce a variety of concepts in a simplified form that is further disclosed in the detailed description of the embodiments. This summary is not intended to identify key or essential inventive concepts of the claimed subject matter, nor is it intended to determine the scope of the claimed subject matter.

The disclosed system may include a clinical education transition coordinator/preceptor/preceptor system configured for cloud-based clinical assessment, training, and competency management of users. The system may additionally include a shared marketplace or consortium, including resources relevant to specific geographic clinical education markets.

The disclosed system may include a clinical education transition coordinator/preceptor/preceptor application configured for training individuals in order to reskill and upskill licensed clinicians. The system may be configured to provide clinical site support for clinical students during clinical rotations.

In some aspects, the system for managing a transition to practice model for clinical reskilling, upskilling, training, and providing a resource for colleges and universities for clinical site placement support includes at least one computing device in operable communication with a user network and an application server in operable communication with the user network to host an application program for matching a student with a preceptor to facilitate clinical reskilling, upskilling, training, and clinical site placement. A matching module receive at least one input from a student and a preceptor and analyzes the at least one input to match the student with the preceptor based on one or more parameters to suggest a suitable placement of the student with the preceptor at a healthcare facility.

In one aspect, the at least one input includes at least one of the following: an availability, at least one qualification, a clinical specialty, an amount of experience, a location, and at least one user preference.

In one aspect, a compliance module receives one or more compliance documents and to ensure compliance to a regulatory standard.

In one aspect, an analysis module analyzes one or more metrics associated with at least one of the following: a student success metric, a facility metric, a preceptor metric.

In one aspect, a communications module enables the transmission, via a network, between the student and the preceptor.

In one aspect, a document management module stores and manages a plurality of documents uploaded by the student and the preceptor.

In one aspect, a display module is in operable communication with a computing device to display a user interface for operating the system.

The system provides a benefit to students, clinicians, preceptors, and healthcare facilities by efficiently matching candidates with potential clinical sites and preceptors. This supports clinician career growth and stability while providing student access to clinical experiences with no expenses for the preceptors. Healthcare facilities may utilize the system to enhance and stabilize their workforce and to improve patient care outcomes.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a system architecture diagram, according to some embodiments;

FIG. 2 illustrates an application program and modules in communication with the computing system, according to some embodiments;

FIG. 3 illustrates a method for matching a student with a preceptor to facilitate clinical reskilling, upskilling, training, and clinical site placement, according to some embodiments;

FIG. 4 illustrates a screenshot of the preceptor communication interface, according to some embodiments;

FIG. 5 illustrates a screenshot of the preceptor communication interface, according to some embodiments;

FIG. 6 illustrates a screenshot of the preceptor milestone interface, according to some embodiments;

FIG. 7 illustrates a screenshot of the preceptor profile interface, according to some embodiments;

FIG. 8 illustrates a screenshot of the preceptor calendar interface, according to some embodiments;

FIG. 9 illustrates a screenshot of the student verification interface, according to some embodiments;

FIG. 10 illustrates a screenshot of the student communication interface, according to some embodiments; and

FIG. 11 illustrates a screenshot of the student profile interface, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The disclosed system may include a clinical education transition coordinator/preceptor/preceptor system configured for cloud-based clinical assessment, training, and competency management of users. The system may additionally include a shared marketplace or consortium, including resources relevant to specific geographic clinical education markets. The system may provide a means for a flat fee-based professional development service that provides expert clinical educators to support both healthcare and academia. The system may be arranged to provide for clinician reskilling and upskilling transition to practice model as well as clinical site support for clinical students during clinical rotations.

In practice and in use, the system may be utilized in cases where healthcare providers have a need for clinical staff in one or more departments. The healthcare provider may access the system on a flat fee-based model. The system may conduct an assessment with the health care provider's human resources team and clinical leadership team to determine how many clinicians are needed and how quickly they need to be placed in a position. The system may be utilized by local for regional colleges or universities for access to the clinical education transition coordinator/preceptors as site adjunct faculty.

A clinical education transition coordinator/preceptor is a registered nurse (or other healthcare professional based on facility need) who works with the healthcare provider to reskill and or upskill clinicians into high-demand clinical specialty areas. In addition, this role is dually aligned with the academic clinical program at the local/regional college or university to provide oversight as an adjunct clinical instructor to ensure the student clinician obtains the clinical experience needed for graduation. Clinical education transition coordinator/preceptors may provide education and evaluation to clinical and patient care staff and facilitates evidenced-based research within a specialized area of healthcare. A clinical education transition coordinator/preceptor collaborates with medical practitioners to incorporate clinical processes into the plan of care for a specialized group of patients. A clinical education transition coordinator/preceptor may perform direct specialty clinical services to patients as required, ensuring adherence to client facility standards and all related policies, procedures, and guidelines. A clinical education transition coordinator/preceptor schedules and supervises students in clinical practice, internships, observations, field experiences, and other similar course and graduation requirements. Debrief and evaluate students in clinical practice, observations, and field experiences, and other similar course and graduation requirements.

In practice and in use, the system may be used in conjunction with healthcare provider leadership staff to identify cohorts for the reskilling or upskilling transition to practice model program. Cohort participants may work with the clinical education transition coordinator/preceptor to orient backfills to their respective roles using a cloud-based pre-assessment, training modules, competency knowledge assessments, and cloud-based competency skills checklists. After training, backfills may be placed on their respective schedules and receive specialty training programs. The clinical education transition coordinator/preceptors may be locally on-site in the department as a clinical resource for the duration of training programs.

In this way, the system may facilitate decreasing contingent staffing costs, increasing internal staff retention and satisfaction, decreasing role vacancy rates, and improving patient care quality.

In some embodiments, the system provides preceptor matching functions. In such, the system utilizes an algorithm to match students with qualified clinical preceptors based on their specific needs, preferences, location, etc. This ensures optimal pairing by considering factors such as clinical specialty, experiences, availability, and the like.

In some embodiments, the system offers means for scheduling clinical rotations, managing student assignments, and tracking progress of the student. Real-time updates and notifications to stakeholders are transmitted which ensures accurate information is disseminated to each party.

In some embodiments, the system facilitates the distribution of financial incentives including stipends to preceptors which are funded through academic program allocations, government workforce innovation grants, consortium memberships, etc. In such, financial processes are streamlined and automated (at least in part) to ensure timely and accurate payments, thus enhancing preceptor engagement and satisfaction.

In some embodiments, the system may enable the monitoring and analysis of performance of students, preceptors, and overall program outcomes.

In some embodiments, the system provides compliance and documentation capabilities to simplify the documentation processes for students, preceptors, and faculty to ensure their adherence to institutional and regulatory standards.

While the example of the medical industry is utilized, it is to be understood that the system can be modified to provide flexibility for use in various industries such as corporate training, mentorship programs, professional development initiatives, etc.

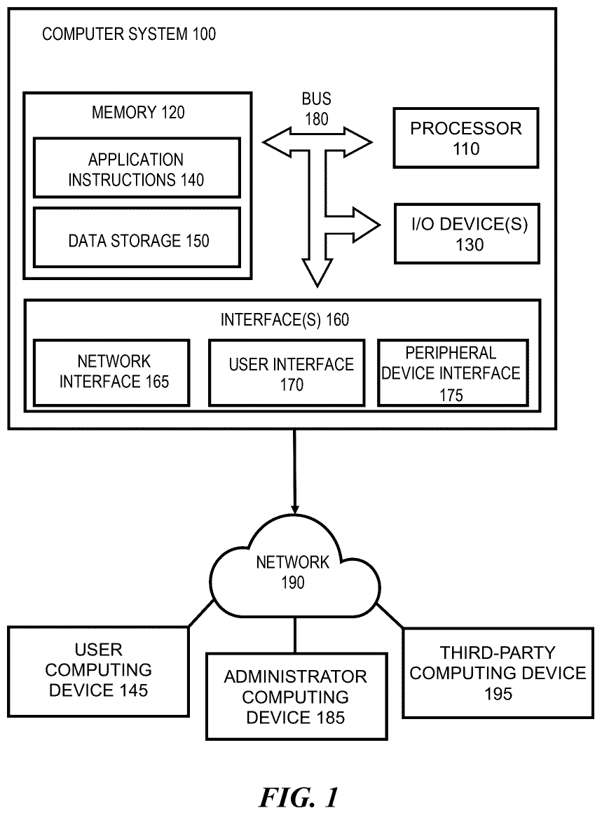

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computing device 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 150, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., Android, C, C++, C#, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a user computing device 145, an administrator computing device 185 and a third-party computing device 195 each in communication via the network 190. The user computing device 145 may be utilized a user (e.g., a healthcare provider) to interact with the various functionalities of the system including to perform patient rounds, handoff patient rounding responsibility, perform biometric verification tasks, and other associated tasks and functionalities of the system. The administrator computing device 185 is utilized by an administrative user to moderate content and to perform other administrative functions. The third-party computing device 195 may be utilized by third parties to receive communications from the user computing device, transmit communications to the user via the network, and otherwise interact with the various functionalities of the system.

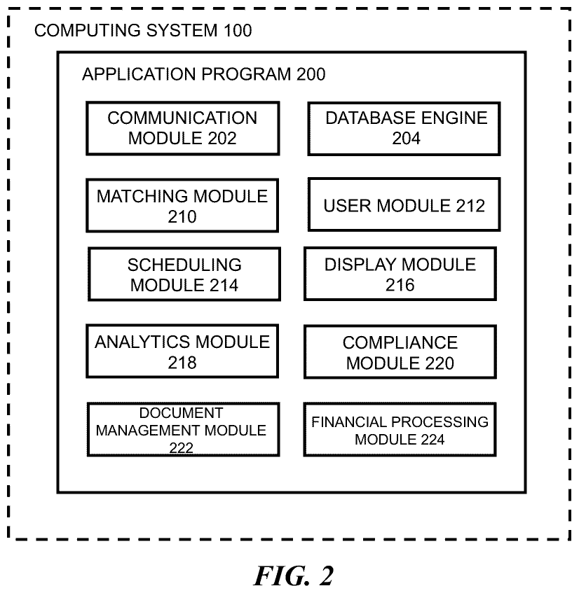

FIG. 2 illustrates an example computer architecture for the application program 200 operated via the computing system 100. The computer system 100 comprises several modules and engines configured to execute the functionalities of the application program 200, and a database engine 204 configured to facilitate how data is stored and managed in one or more databases. In particular, FIG. 2 is a block diagram showing the modules and engines needed to perform specific tasks within the application program 200.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of a communication module 202, a database engine 204, a matching module 210, a user module 212, a scheduling module 214, a display module 216, an analytics module 218, a compliance module 220, a document management module 222, and a financial processing module 224.

In some embodiments, the communication module 202 is configured for receiving, processing, and transmitting a user command and/or one or more data streams. In such embodiments, the communication module 202 performs communication functions between various devices, including the user computing device 145, the administrator computing device 185, and a third-party computing device 195. In some embodiments, the communication module 202 is configured to allow one or more users of the system, including a third-party, to communicate with one another. In some embodiments, the communications module 202 is configured to maintain one or more communication sessions with one or more servers, the administrative computing device 185, and/or one or more third-party computing device(s) 195.

In some embodiments, the communication module 202 enables for students, preceptors, and other users to communication via video messaging, audio messaging, text, conferencing, and the like.

In some embodiments, a database engine 204 is configured to facilitate the storage, management, and retrieval of data to and from one or more storage mediums, such as the one or more internal databases described herein. In some embodiments, the database engine 204 is coupled to an external storage system. In some embodiments, the database engine 204 is configured to apply changes to one or more databases. In some embodiments, the database engine 204 comprises a search engine component for searching through thousands of data sources stored in different locations.

In some embodiments, the database engine 204 is in communication with a document management module 222 in order to receive and store documents related to each user. The document management module 222 may enable users to upload documents which are stored via the database engine 204.

In some embodiments, the matching module 210 analyzes the user profiles of the students and preceptors, taking into account factors such as location, specialty, availability, and other preferences. The matching module 210 then matches students with preceptors based on these inputs, program requirements, and other factors. In such, the matching module 210 may output a list of suggested matches to the user based on their inputs.

In some embodiments, the user module 212 facilitates the creation of a user account for the application system. The user module 212 may allow the user to create a user profile which includes user information, user preferences, and user-associated information. The user module may also be used to assign a user type to each user. User types may include preceptors, students, clinicians, healthcare administrators, etc.

In some embodiments, the scheduling module 214 is in operable communication with the computing device to enable the input of schedules for each user. Schedules may be used for various functions of the system including matching, scheduling meetings, inputting availability, scheduling assessments, etc.

In some embodiments, the display module 216 is configured to display one or more graphic user interfaces, including, e.g., one or more user interfaces, one or more consumer interfaces, one or more video presenter interfaces, etc. In some embodiments, the display module 216 is configured to temporarily generate and display various pieces of information in response to one or more commands or operations. The various pieces of information or data generated and displayed may be transiently generated and displayed, and the displayed content in the display module 216 may be refreshed and replaced with different content upon the receipt of different commands or operations in some embodiments. In such embodiments, the various pieces of information generated and displayed in a display module 216 may not be persistently stored.

In some embodiments, the analytics module 218

In some embodiments, the compliance module 220 facilitates the completion and transmission of documents which are required based on regulatory requirements. This may include ensuring the user has completed necessary evaluations, feedback forms, and performance assessments. The compliance module may also facilitate the uploading and storing of documents which relate to compliance.

In some embodiments, the financial processing module 224 may autonomously, or semi-autonomously process payments which are sent to various parties.

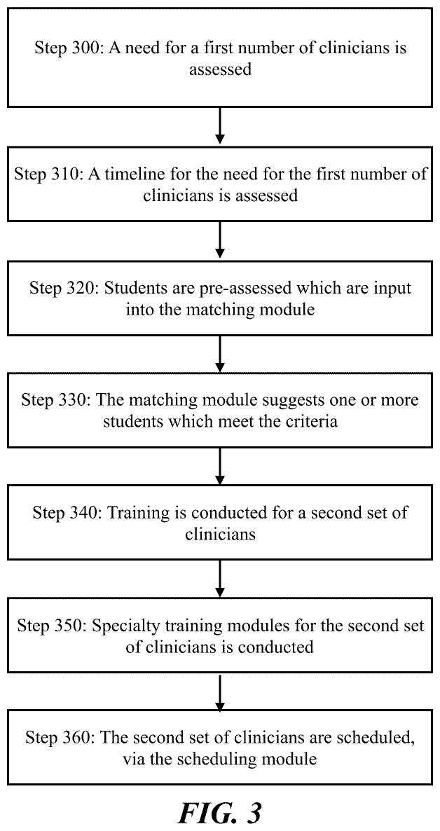

FIG. 3 illustrates a method for managing a transition to practice model for clinical reskilling, upskilling, training and providing a resource for colleges and universities for clinical site placement support. In step 300, a need for a first number of clinicians is assessed and in step 310, a timeline for the need for the first number of clinicians is assessed. In step 320, a plurality of students (i.e., candidates) are pre-assessed which are input into the matching module. In step 330, the matching module suggests one or more students which meet the criteria. In step 340, training is conducted for a second set of clinicians and in step 350, specialty training modules for the second set of clinicians with the support of the system is conducted. In step 360, the second set of clinicians are scheduled, via the scheduling module.

As mentioned hereinabove, the system provides a process for matching students with clinical preceptors, managing clinical placements, and supporting the operational needs of academic institutions and healthcare providers. Once the user has registered with the system, an account is created based on their user type (e.g., clinical preceptor, student, clinician, etc.) and the user enters their personal information, professional credentials, availability and other preferences. The system may then verify the user's credentials and information provided by the user through integrated checks or manual review by administrators.

The matching module may then analyze the user profiles of the students and preceptors, taking into account factors such as location, specialty, availability, and other preferences. The matching module then matches students with preceptors based on these inputs, program requirements, and other factors.

Users may then manage their schedules, via the scheduling module including availability for critical placements and other commitments.

The communications module enables the users to transmit communications and notifications to other users of the system. Communications may include video conferencing, messaging, e-mails, alerts, and other forms of digital communications.

The compliance module facilitates the completion and transmission of documents which are required based on regulatory requirements. This may include ensuring the user has completed necessary evaluations, feedback forms, and performance assessments. The compliance module may also facilitate the uploading and storing of documents which relate to compliance.

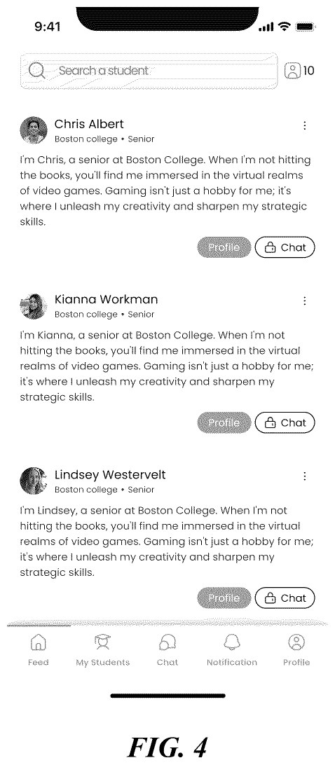

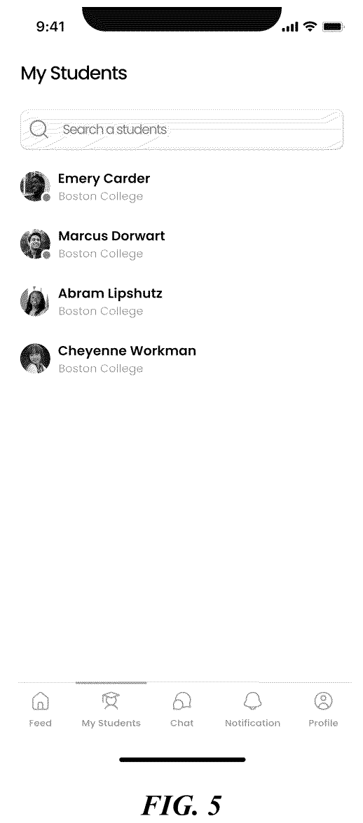

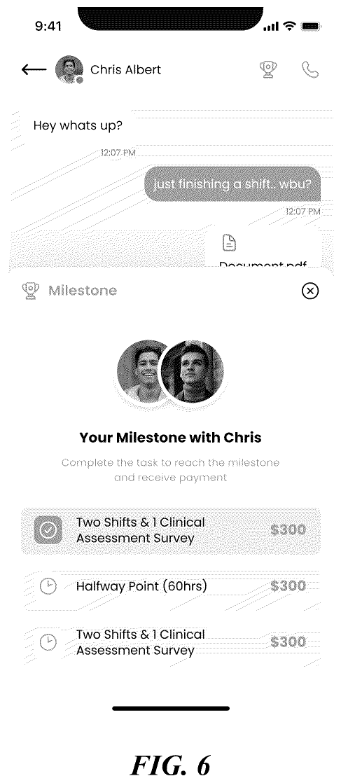

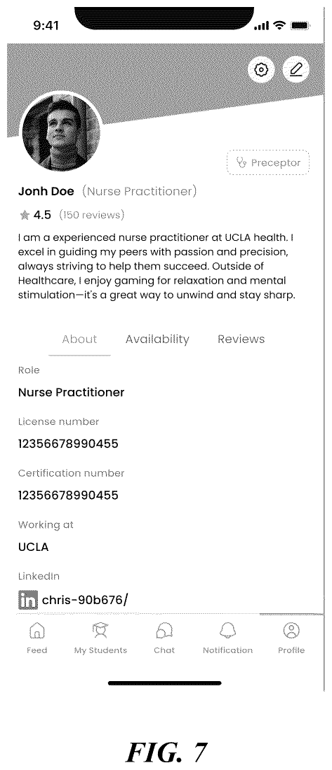

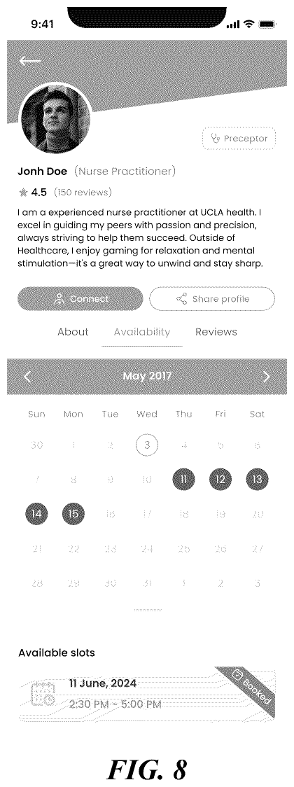

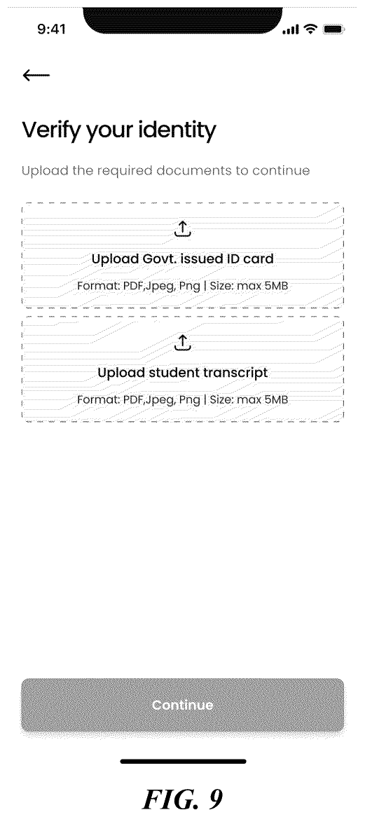

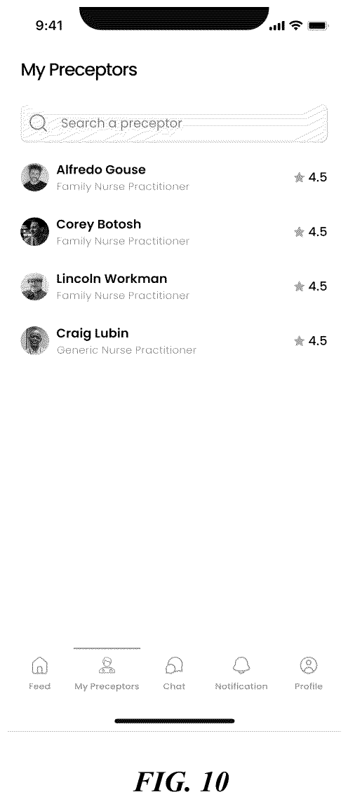

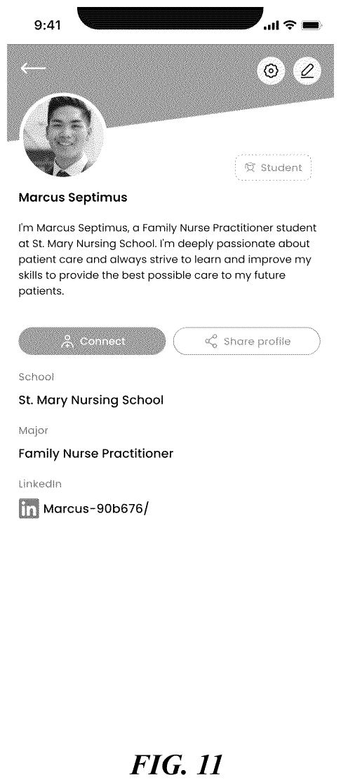

FIGS. 4-11 illustrate exemplary screenshots of user interfaces which are displayed to the users including the preceptor and/or students. In particular, FIGS. 4-8 illustrate the preceptor user flow which is accessible by preceptors and similar users of the system. Preceptors may utilize the interfaces to communicate with other users (e.g., students, other preceptors, administrators, and healthcare personnel). Further, the preceptors may utilize the interface to create and view user profiles associated with preceptors and/or students, as well as to verify their identity. Preceptors may also utilize the interface to submit a performance review for students they have interacted with, to view and indicate the completion of milestones, etc. FIGS. 9-11 illustrate the student user flow and interfaces associated therewith. Students may utilize the user interface to verify their identity, upload documents, create a user profile, submit a review for a preceptor or preceptors they have interacted with, and communicate with other users of the system.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrase “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

It will be appreciated by persons skilled in the art that the present embodiment is not limited to what has been particularly shown and described hereinabove. A variety of modifications and variations are possible considering the above teachings without departing from the following claims.

I/We claim:

1. A method for managing a transition to practice model for clinical reskilling, upskilling, training, and providing a resource for colleges and universities for clinical site placement support, the method comprising the steps of:
assessing a need for a first number of clinicians;
assessing a timeline for the need for the first number of clinicians;
pre-assessing candidates for the need for the first number of clinicians;
conducting one or more training modules for a second set of clinicians;
conducting one or more specialty training modules for the second set of clinicians with the utilization of a clinical education transition coordinator/preceptor; and
scheduling the second set of clinicians.

2. The method of claim 1, further comprising a matching module to match one or more preceptors to one or more students.

3. The method of claim 2, wherein the matching module performs the steps of:
receiving at least one input from each of the one or more students;
receiving at least one input from each of the one or more preceptors;
analyzing the at least one input and matching one or more parameters associated with the at least one input from the one or more students to the at least one input received from the one or more preceptors to match at least one student to at least one preceptor.

4. The method of claim 3, wherein the one or more user inputs includes at least one of the following: an availability, at least one qualification, a clinical specialty, an amount of experience, a location, and at least one user preference.

5. The method of claim 1, further comprising a compliance module to receive one or more compliance documents and to ensure compliance to a regulatory standard.

6. The method of claim 1, further comprising an analysis module to analyze one or more metrics associated with at least one of the following: a student success metric, a facility metric, a preceptor metric.

7. The method of claim 1, further comprising a communications module to enable the transmission, via a network, between the student and the preceptor.

8. The method of claim 1, further comprising a document management module to store and manage a plurality of documents uploaded by the student and the preceptor.

9. The method of claim 1, further comprising a display module in operable communication with a computing device to display a user interface for operating the system.

10. A system for managing a transition to practice model for clinical reskilling, upskilling, training, and providing a resource for colleges and universities for clinical site placement support, the system comprising:
at least one computing device in operable communication with a user network;
an application server in operable communication with the user network, the application server configured to host an application program for matching a student with a preceptor to facilitate clinical reskilling, upskilling, training, and clinical site placement;
a matching module to receive at least one input from a student and a preceptor, and to analyze the at least one input to match the student with the preceptor based on one or more parameters to suggest a suitable placement of the student with the preceptor at a healthcare facility.

11. The system of claim 10, wherein the at least one inputs includes at least one of the following: an availability, at least one qualification, a clinical specialty, an amount of experience, a location, and at least one user preference.

12. The system of claim 10, further comprising a compliance module to receive one or more compliance documents and to ensure compliance to a regulatory standard.

13. The system of claim 10, further comprising an analysis module to analyze one or more metrics associated with at least one of the following: a student success metric, a facility metric, a preceptor metric.

14. The system of claim 10, further comprising a communications module to enable the transmission, via a network, between the student and the preceptor.

15. The system of claim 10, further comprising a document management module to store and manage a plurality of documents uploaded by the student and the preceptor.

16. The system of claim 10, further comprising a display module in operable communication with a computing device to display a user interface for operating the system.

17. A system for managing a transition to practice model for clinical reskilling, upskilling, training, and providing a resource for colleges and universities for clinical site placement support, the system comprising:
at least one computing device in operable communication with a user network;
an application server in operable communication with the user network, the application server configured to host an application program for matching a student with a preceptor to facilitate clinical reskilling, upskilling, training, and clinical site placement;
a matching module to perform the following steps:
assessing a need for a first number of clinicians;
assessing a timeline for the need for the first number of clinicians;
pre-assessing candidates for the need for the first number of clinicians;
conducting one or more training modules for a second set of clinicians;
conducting one or more specialty training modules for the second set of clinicians with the utilization of a clinical education transition coordinator/preceptor; and
scheduling the second set of clinicians.

18. The system of claim 17, further comprising a matching interface provided by the display module on a display of the computing device, the matching interface to enable viewing of a match between the student and the preceptor.

19. The system of claim 18, further comprising a document input interface to enable the uploading of one or more documents, wherein a compliance module ensures the compliance of the one or more documents in relation to one or more regulatory standards.

20. The system of claim 19, further comprising an analysis module to analyze a plurality of student metrics and a plurality of preceptor metrics.
